(19) United States
(12) Patent Application Publication
(10) Pub. No.: US 2026/0191921 A1
(43) Pub. Date: Jul. 9, 2026

(54) PHARMACEUTICAL COMPOSITION OF NON-ENVELOPED VIRUS

(71) Applicant: JOINT STOCK COMPANY "BIOCAD", Saint Petersburg (RU)

(72) Inventors: Dmitrii Aleksandrovich TOLSTYKH, Saint Petersburg (RU); Aleksandra Aleksandrovna SOZONOVA, Saint Petersburg (RU); Ekaterina Aleksandrovna LOMKOVA, Saint Petersburg (RU); Lina Igorevna FEDORENKO, g.Bronnitsy (RU); Galina Sergeevna NAGIBINA, Tugulymsky r-n (RU); Dmitry Valentinovich MOROZOV, Saint Petersburg (RU)

(21) Appl. No.: 19/132,543

(22) PCT Filed: Nov. 23, 2023
(86) PCT No.: PCT/RU2023/050271

§ 371 Date: May 23, 2025

(30) Foreign Application Priority Data
Nov. 24, 2022 (RU) 2022130458

(51) Int. Cl. A61K 35/76; A61K 9/08; A61K 47/02; A61K 47/10; A61K 47/18; A61K 48/00; C12N 15/864

(52) U.S. Cl. CPC A61K 35/76; A61K 9/08; A61K 47/02; A61K 47/10; A61K 47/183; A61K 48/00; C12N 15/8645; C12N 2750/14132; C12N 2750/14143

(57) ABSTRACT

The present invention relates to the field of pharmaceuticals, gene therapy and medicine, specifically to pharmaceutical compositions of a vector based on recombinant non-enveloped virus, in particular recombinant adeno-associated virus (rAAV), which compositions may be used for the treatment and prophylaxis of various diseases.

### FIELD OF THE INVENTION

The present invention relates to the field of pharmaceuticals, gene therapy and medicine, specifically to pharmaceutical compositions of a vector based on recombinant non-enveloped virus, in particular recombinant adeno-associated virus (rAAV), which compositions may be used for the treatment and prophylaxis of various diseases.

### BACKGROUND OF THE INVENTION

Typical representatives of non-enveloped viruses are parvoviruses, noroviruses, as well as rotaviruses and adenoviruses.

Simple or non-enveloped viruses consist of a nucleic acid and a protein envelope called a capsid. The capsid consists of repeating morphological subunits called capsomers. The nucleic acid and the capsid interact with one another to form a nucleocapsid. In each virus, oligomerization of capsid proteins during capsid assembly typically leads to a certain type of symmetrical quaternary structure. Most viruses have capsids of a spiral or icosahedral structure (Lidmar J, Mirny L, Nelson Dr. Virus shapes and buckling transitions in spherical shells. Phys Rev E Stat Nonlin Soft Matter Phys. 2003 November; 68(5 Pt 1): 051910. doi: 10.1103/PhysRevE.68.051910. Epub 2003 Nov. 25. PMID: 14682823).

Unlike non-enveloped viruses, the capsid of enveloped viruses is covered by a lipid membrane known as a supercapsid. The envelope is acquired by the capsid from an intracellular membrane in the virus' host; examples include the inner nuclear membrane, the Golgi apparatus membrane, and the cell's outer membrane. The presence of a lipid envelope makes viruses less resistant to physical and chemical stresses.

The capsid of non-enveloped viruses performs the function of protecting the genome during long-term storage, as well as from chemical and physical stresses, such as UV radiation, extreme pH or temperatures, proteolytic and nucleolytic agents. Non-enveloped viruses may retain their properties in a dried state for a long time on various surfaces, both porous and non-porous (Abad F X, Pinto R M, Bosch A. Survival of enteric viruses on environmental fomites. Appl Environ Microbiol. 1994 October; 60(10): 3704-10. doi: 10.1128/aem.60.10.3704-3710.1994. PMID: 7986043; PMCID: PMC201876).

Viruses introduce genetic material thereof into the genome of a host cell, so it was proposed to use them as vectors for delivering genetic information to cells. Vectors based on retrovirus, adenovirus, adeno-associated virus (AAV), herpes simplex virus, and the like are employed for therapeutic purposes. Among these viruses, adenovirus and AAV refer to non-enveloped viruses.

Vectors based on recombinant AAV and recombinant adenovirus are currently the most widely used and developed gene therapy products, and they are also used in vaccine development.

To date, gene therapy is considered a potentially universal approach to treatment of wide spectrum of diseases: infectious diseases, genetic diseases, malignant neoplasms, and the like.

Viruses or viral vectors (viral particles) must retain structural integrity thereof in order to exert infectious and biological activity during production, transportation, storage, and use. The structural integrity of a viral vector may be destructed during production, transportation, storage, and use of vaccines or products comprising the viral vector, thus excluding usage thereof as a delivery vector.

The prior art provides liquid pharmaceutical compositions of AAV (WO2018/128689, WO2019/094253, WO2020/014479, WO2020/214929), liquid pharmaceutical compositions of adenovirus (WO00/29024, WO2017/013169).

Despite this, there is still a need for improved highly stable pharmaceutical compositions comprising a vector based on recombinant non-enveloped virus.

### BRIEF DESCRIPTION OF DRAWINGS

FIG. 1 is a graph showing changes in the particle size of the vector based on adeno-associated virus serotype 9 as a function of time in test formulations under 65° C. thermal stress.

HisArg, His, Tris, PBS, PhosCitr-pharmaceutical compositions HisArg AAV9, His AAV9, Tris AAV9, PBS AAV9, PhosCitr AAV9, respectively, shown in Table 9.

FIG. 2 is a graph showing changes in the particle size of the vector based on adeno-associated virus serotype 5 as a function of time in test formulations under 40° C. thermal stress.

HisArg, His, Tris, PBS, PhosCitr-pharmaceutical compositions HisArg AAV5, His AAV5, Tris AAV5, PBS AAV5, PhosCitr AAV5, respectively, shown in Table 18.

### DETAILED DESCRIPTION OF THE INVENTION

The authors of the invention surprisingly produced stable pharmaceutical compositions comprising a vector based on recombinant non-enveloped virus, in particular recombinant adeno-associated virus (rAAV), which have increased colloidal stability (higher aggregation temperature), stability to DNA retention (higher DNA extraction temperature), providing for stability while storage in frozen form as well as in liquid form (no titer drop while storage at (5±3° C.), and reduced titer drop under thermal stress). The developed pharmaceutical compositions may be used for treatment and prophylaxis of various diseases.

### Definitions

Unless defined otherwise herein, all technical and scientific terms used in connection with the present invention will have the same meaning as is commonly understood by those skilled in the art.

Furthermore, unless otherwise required by context, singular terms shall include plural terms, and the plural terms shall include the singular terms.

As used in the present description and claims that follow, unless otherwise dictated by the context, the words “include” and “comprise”, or variations thereof such as “includes”, “including”, “comprises”, or “comprising”, will be understood to imply the inclusion of a stated integer or group of integers but not the exclusion of any other integer or group of integers.

The term “pharmaceutical composition” refers to a composition and/or formulation comprising a vector based on recombinant non-enveloped virus in a therapeutically effective amount and excipients or auxilliary substances (carriers, diluents, fillers, solvents, and the like), the choice and proportions of which depend on the type and route of administration and dosage.

The term “aqueous composition” as used herein refers to a water-based composition, the water in the composition may be: water, water for injections, physiologic saline (0.9%-1.0% aqueous solution of sodium chloride).

The term “lyophilized” as used herein refers to a formulation that has been subjected to a process known in the art as freeze-drying, which includes freezing the formulation followed by removal of ice from the frozen contents.

A pharmaceutical composition is “stable” if the active agent retains physical stability and/or chemical stability and/or biological activity thereof during the specified shelf life at storage temperature, for example, at (5±3° C.) Further, the active agent may retain both physical and chemical stability, as well as biological activity. Storage period is adjusted based on the results of stability test in accelerated or natural aging conditions.

The term “vector” as used herein means a nucleic acid molecule capable of transporting another nucleic acid to which it has been linked. Furthermore, the term “vector” herein refers to a recombinant viral particle capable of transporting a nucleic acid.

The term “long-term storage” or “long term stability” should be understood to mean that a pharmaceutical composition may be stored for three months or more, for six months or more, for one year or more, and the composition may have a minimum stable shelf life of at least two years as well. Generally speaking, the terms “long term storage” and “long term stability” further include stable storage durations that are at least comparable to or better than the stable shelf life typically required for currently available commercial formulations, without losses in stability that would render the formulation unsuitable for its intended pharmaceutical application.

The term “buffering agent” refers to an acid or base component (typically a weak acid or weak base) of the buffer or buffer solution. A buffering agent helps to maintain the pH value of a solution at or near to a pre-determined value, and the buffering agents are generally chosen to achieve a pre-determined value. A buffering agent may be a single compound which gives rise to a desired buffering effect, especially when said buffering agent is mixed with (and suitably capable of proton exchange with) an appropriate amount (depending on the pre-determined value desired) of corresponding “acid/base conjugate” thereof.

The term “buffer” or “buffer solution” refers to an aqueous solution comprising a mixture of an acid (typically a weak acid, such as e.g. acetic acid, citric acid) and a conjugated base thereof (such as e.g. an acetate or citrate salt, e.g. sodium acetate, sodium citrate, as well as hydrates of said salts, e.g. sodium acetate trihydrate) or alternatively a mixture of a base (typically a weak base, e.g. histidine) and a conjugated acid thereof (e.g. histidine hydrochloride or histidine hydrochloride monohydrate or L-histidine hydrochloride (h/c) monohydrate (m/h) or L-histidine h/c m/h or histidine h/c m/h). The pH value of a “buffer solution” changes only slightly upon addition thereto of a small quantity of strong base or strong acid, as well as upon dilution or concentration due to the “buffering effect” imparted by a “buffering agent”.

Typically, amino acids are L-amino acids. For example, if histidine and histidine hydrochloride monohydrate are used, it is typically L-histidine and L-histidine hydrochloride monohydrate. For example, if arginine is used, it is typically L-arginine. Amino acid equivalents, for example, pharmaceutically acceptable proline salts (for example, proline hydrochloride) may also be used.

### Abbreviations

AAV—adeno-associated virus
BHQ-1—fluorescence extinguisher used in PCR
Ct—cycle threshold
FAM—carboxyfluorescein dye
GFP—green fluorescent protein
GOI—gene of interest
MOI—multiplicity of infection (number of viral particles per cell)
rAAV—recombinant adeno-associated virus
TU—transduction units
Vg—viral genomes
PCR—polymerase chain reaction
ELISA—enzyme-linked immunosorbent assay
FA—functional activity
SE HPLC—size-exclusion high-performance liquid chromatography

The present invention discloses stable pharmaceutical compositions of a vector based on recombinant non-enveloped virus, in particular recombinant adeno-associated virus (rAAV), which compositions may be used for treatment and prophylaxis of various diseases.

In one aspect, the present invention relates to a pharmaceutical composition of a vector based on recombinant non-enveloped virus, the composition comprising:
(i) a vector based on recombinant non-enveloped virus,
(ii) histidine,
(iii) arginine,
(iv) sodium chloride,
(v) magnesium chloride,
(vi) a surfactant, and
(vii) water for injection.

In some embodiments of the invention, histidine is present at a concentration of 2.0-3.58 mg/ml, or 2.3-3.2 mg/ml, or 2.6-3.0 mg/ml, or 2.7-2.8 mg/ml, or 2.79 mg/ml.

In some embodiments of the invention, arginine is present at a concentration of 0.248-0.448 mg/ml, or 0.28-0.416 mg/ml, or 0.3-0.396 mg/ml, or 0.340-0.350 mg/ml, or 0.348 mg/ml.

In some embodiments of the invention, sodium chloride is present at a concentration of 8.0-15.0 mg/ml, or 9.5-13.5 mg/ml, or 10.5-12.5 mg/ml, or 11.0-12.0 mg/ml, or 11.7 mg/ml.

Magnesium chloride may be used both in the form of anhydrous magnesium chloride and in the form of hydrates thereof. In some embodiments of the invention, magnesium chloride is magnesium chloride hexahydrate.

In some embodiments of the invention, magnesium chloride is present at a concentration of 0.15-0.50 mg/ml, or 0.16-0.35 mg/ml, or 0.18-0.25 mg/ml, 0.200-0.210 mg/ml or 0.203 mg/ml.

Any pharmaceutically acceptable surfactant may be used as a surfactant. Surfactants are well known in the art and include, without limitation, polysorbate 20, polysorbate 80, various poloxamers and pluronics, as well as mixtures thereof.

In some embodiments of the invention, the surfactant is present at a concentration of 0.01-1.0 mg/ml, or 0.01-0.5 mg/ml, or 0.01-0.2 mg/ml, or 0.03-0.15 mg/ml, or 0.05 mg/ml, or 0.1 mg/ml.

In some embodiments of the invention, the pharmaceutical composition comprises:

| (i) | a vector based on recombinant | | |
|---|---|---|---|
| | non-enveloped virus, | | |
| (ii) | histidine | 2.0-3.58 | mg/ml, |
| (iii) | arginine | 0.248-0.448 | mg/ml, |
| (iv) | sodium chloride | 8.0-15.0 | mg/ml, |
| (v) | magnesium chloride | 0.15-0.50 | mg/ml, |

| | | |
|---|---|---|
| (vi) | a surfactant | 0.01-1.0 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.3-3.2 | mg/ml, |
| (iii) | arginine | 0.28-0.416 | mg/ml, |
| (iv) | sodium chloride | 9.5-13.5 | mg/ml, |
| (v) | magnesium chloride | 0.16-0.35 | mg/ml, |

| | | |
|---|---|---|
| (vi) | a surfactant | 0.01-0.2 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.6-3.0 | mg/ml, |
| (iii) | arginine | 0.3-0.396 | mg/ml, |
| (iv) | sodium chloride | 10.5-12.5 | mg/ml, |
| (v) | magnesium chloride | 0.18-0.25 | mg/ml, |

| | | |
|---|---|---|
| (vi) | a surfactant | 0.03-0.15 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.7-2.8 | mg/ml, |
| (iii) | arginine | 0.340-0.350 | mg/ml, |
| (iv) | sodium chloride | 11.0-12.0 | mg/ml, |
| (v) | magnesium chloride | 0.200-0.210 | mg/ml, |

| | | |
|---|---|---|
| (vi) | a surfactant | 0.03-0.15 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride | 0.203 | mg/ml, |
| (vi) | a surfactant | 0.05 | mg/ml, |

| | | |
|---|---|---|
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride | 0.203 | mg/ml, |
| (vi) | a surfactant | 0.1 | mg/ml, |

| | | |
|---|---|---|
| (vii) | water for injection | ad 1.0 ml. |

In some embodiments of the invention, magnesium chloride is magnesium chloride hexahydrate.

In some embodiments of the invention, the surfactant is poloxamer 188.

In some embodiments of the invention, the pharmaceutical composition comprises:

| (i) | a vector based on recombinant | | |
|---|---|---|---|
| | non-enveloped virus, | | |
| (ii) | histidine | 2.0-3.58 | mg/ml, |
| (iii) | arginine | 0.248-0.448 | mg/ml, |
| (iv) | sodium chloride | 8.0-15.0 | mg/ml, |
| (v) | magnesium chloride hexahydrate | 0.15-0.50 | mg/ml, |

| | | |
|---|---|---|
| (vi) | a surfactant being poloxamer 188 | 0.01-1.0 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.3-3.2 | mg/ml, |
| (iii) | arginine | 0.28-0.416 | mg/ml, |
| (iv) | sodium chloride | 9.5-13.5 | mg/ml, |
| (v) | magnesium chloride hexahydrate | 0.16-0.35 | mg/ml, |

| | | |
|---|---|---|
| (vi) | a surfactant being poloxamer 188 | 0.01-0.2 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.6-3.0 | mg/ml, |
| (iii) | arginine | 0.3-0.396 | mg/ml, |
| (iv) | sodium chloride | 10.5-12.5 | mg/ml, |
| (v) | magnesium chloride hexahydrate | 0.18-0.25 | mg/ml, |

| | | |
|---|---|---|
| (vi) | a surfactant being poloxamer 188 | 0.03-0.15 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.7-2.8 | mg/ml, |
| (iii) | arginine | 0.340-0.350 | mg/ml, |
| (iv) | sodium chloride | 11.0-12.0 | mg/ml, |
| (v) | magnesium chloride hexahydrate | 0.200-0.210 | mg/ml, |

| | | |
|---|---|---|
| (vi) | a surfactant being poloxamer 188 | 0.03-0.15 mg/ml, and |
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride hexahydrate | 0.203 | mg/ml, |
| (vi) | a surfactant being poloxamer 188 | 0.05 | mg/ml, |

| | | |
|---|---|---|
| (vii) | water for injection | ad 1.0 ml. |

In some embodiments of the invention, the pharmaceutical composition comprises:

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride hexahydrate | 0.203 | mg/ml, |
| (vi) | a surfactant being poloxamer 188 | 0.1 | mg/ml, |

| | | |
|---|---|---|
| (vii) | water for injection | ad 1.0 ml. |

In some embodiments of the invention, histidine is L-histidine.

In some embodiments of the invention, arginine is L-arginine.

In some embodiments of the invention, the pharmaceutical composition has pH 7.0-9.0, 7.5-8.5 or 7.7-8.7, or 8.0, or 8.2.

In some embodiments of the invention, the above pharmaceutical compositions are suitable for lyophilization, i.e. they can act as a pre-lyophilization solution.

In one aspect, the present invention relates to a pharmaceutical composition of a vector based on recombinant non-enveloped virus, which composition is provided in dry (i.e. powder or granular) form to be dissolved in a suitable solvent (e.g. water) prior to administration. Such pharmaceutical composition may be prepared by, for example, lyophilisation, i.e. a process which is known in the art as freeze drying, which involves freezing a product followed by removal of solvent from the frozen contents.

In one aspect, the present invention relates to a lyophilized pharmaceutical composition of a vector based on recombinant non-enveloped virus, said composition being produced by lyophilizing any pharmaceutical composition described above. Accordingly, the pharmaceutical compositions according to the present invention may be either aqueous pharmaceutical compositions or lyophilized pharmaceutical compositions (lyophilizates).

The described pharmaceutical compositions are suitable for delivering therapeutic agents to subjects for treatment or prophylaxis of various diseases or disorders. They may be used in gene therapy for treatment of diseases such as hemophilia A, hemophilia B, malignant neoplasms, spinal muscular atrophy, and the like, as well as in the development of vaccines for prophylaxis of, for example, infectious diseases.

The pharmaceutical compositions according to the invention may comprise the vector based on recombinant non-enveloped virus at various concentrations. The concentration of the viral vector may depend, for example, on the disease for prophylaxis or treatment of which the above pharmaceutical compositions will be used, as well as on the age, weight and state of health of the patient, and thus may vary from patient to patient. A suitable dose can be adjusted by the decision of the attending physician so that it can be administered to the patient once or through several injections.

In some embodiments of the invention, the vector based on recombinant non-enveloped virus is present at a concentration of 1.0*10⁵-1.0*10¹⁴ viral genomes/ml, or 1.0*10⁹-1.0*10¹⁴ viral genomes/ml, or 1.0*10⁹-5.0*10¹³ viral genomes/ml, or 1.0*10⁹-1.0*10¹³ viral genomes/ml, or (1.0±0.3)*10⁵ viral genomes/ml, or (2.0±0.6)*10⁵ viral genomes/ml, (5.0±1.5)*10⁵ viral genomes/ml, or (1.0±0.3)*106 viral genomes/ml, or (2.0±0.6)*106 viral genomes/ml, (5.0±1.5)*106 viral genomes/ml, or (1.0±0.3)*107 viral genomes/ml, or (2.0±0.6)*107 viral genomes/ml, (5.0±1.5)*107 viral genomes/ml, or (1.0±0.3)*108 viral genomes/ml, or (2.0±0.6)*108 viral genomes/ml, (5.0±1.5)*108 viral genomes/ml, or (1.0±0.3)*10⁹ viral genomes/ml, or (2.0±0.6)*10⁹ viral genomes/ml, (5.0±1.5)*10⁹ viral genomes/ml, or (1.0±0.3)*10¹⁰ viral genomes/ml, or (2.0±0.6)*10¹⁰ viral genomes/ml, (5.0=1.5)*10¹⁰ viral genomes/ml, or (1.0±0.3)*10¹¹ viral genomes/ml, or (2.0±0.6)*10¹¹ viral genomes/ml, (5.0±1.5)*10¹¹ viral genomes/ml, or (1.0±0.3)*10¹² viral genomes/ml, or (2.0=0.6)*10¹² viral genomes/ml, (5.0±1.5)*10¹² viral genomes/ml, or (1.0±0.3)*10¹³ viral genomes/ml, or (2.0±0.6)*10¹³ viral genomes/ml, (5.0±1.5)*10¹³ viral genomes/ml, or (1.0±0.3)*10¹⁴ viral genomes/ml.

The recombinant non-enveloped viruses in the above pharmaceutical compositions may be rAAV, recombinant adenoviruses and other types of recombinant non-enveloped viruses.

The vector based on recombinant adenovirus may be produced using any species, strain, serotype or any combination of species, strains or serotypes of adenovirus or chimeric adenovirus. Human adenovirus serotypes include any one of serotypes 2, 4, 5, 7, 11, 26, 34, 35, 36, 48, 49 or 50 or combinations thereof, derivatives, modifications or pseudotypes.

In some embodiments of the invention, the vector based on recombinant non-enveloped virus is a vector based on rAAV. It may be produced using any strain, AAV serotype (for example, AAV1, AAV2, AAV3, AAV4, AAV5, AAV6, AAV7, AAV8, AAV9, AAV10, AAV11, AAV12, AAV13, AAV14, AAV15 and AAV16) or any combination of strains, serotypes (for example, a vector based on rAAV that includes two or more serotypes); may contain an AAV capsid protein (capsid) of any serotype (AAV1, AAV2, AAV3, AAV4, AAV5, AAV6, AAV7, AAV8, AAV9, AAV10, AAV11, AAV12, AAV13, AAV14, AAV15 and AAV16) or combinations thereof, derivatives, modifications or pseudotypes.

In some embodiments of the invention, the vector based on rAAV comprises an AAV5 or AAV6 or AAV9 capsid.

In some embodiments of the invention, the AAV5 or AAV6 or AAV9 capsid may be a modified capsid.

The vector based on rAAV may be modified genetically and/or chemically.

The vector based on rAAV may be genetically modified to produce vectors based on rAAV with altered receptor usage, antigenicity, transduction efficiency and/or tissue tropism, and to insert peptide ligands, antibodies, antibody fragments, MHC (major histocompatibility complex) and/or receptors into the viral capsid. For example, the vector based on rAAV may be genetically modified by introducing one or more amino acid mutations, such as point mutations.

The phrase “more point mutations” refers to two, three, four, five, six, seven, eight, nine, or ten point substitutions.

Particularly preferred embodiments include substitutions (mutations) that are conservative in nature, i.e. substitutions that take place within a family of amino acids that are joined in their side chains. In particular, amino acids are typically divided into four families: (1) acidic amino acids are aspartate and glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. Phenylalanine, tryptophan, and tyrosine are sometimes classified as aromatic amino acids. For example, it is reasonably predictable that an isolated substitution of leucine for isoleucine or valine, an aspartate for a glutamate, a threonine for a serine, or a similar conservative substitution of an amino acid for a structurally related amino acid, will not have a major effect on the biological activity. For example, the polypeptide of interest may include up to about 5-10 conservative or non-conservative amino acid substitutions, so long as the desired function of the molecule remains intact.

A variant of point mutations in the sequences of AAV proteins VP1, VP2, or VP3 using amino acid substitutions is a substitution of at least one amino acid residue in the AAV protein VP1, VP2, or VP3 with another amino acid residue.

Conservative substitutions are shown in Table A under “preferred substitutions”.

TABLE A

| Initial | | Preferred |
|---|---|---|
| residue | Exemplary substitutions | substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

For example, the international application WO2012/145601 describes adeno-associated virus (AAV) virions with variant capsid protein, where the AAV virions exhibit greater infectivity of retinal cells, when administered via intravitreal injection, compared to wild-type AAV. The international application WO2013/158879 describes an adeno-associated virus (AAV) vector for delivering to a subject a heterologous nucleic acid sequence comprising the capsid protein VP1 comprising one or more lysine substitutions, wherein one lysine substitution is K137R, wherein said lysine substitution is effective for inhibiting ubiquitination of said capsid protein, thereby increasing transduction efficiency of said AVV vector in target cells.

The vector based on rAAV may be chemically modified to alter tissue tropism. Also, chemically modified vectors based on rAAV may exhibit altered receptor usage, antigenicity, transduction efficiency, and/or tissue tropism. Chemically modified vectors based on rAAV are created by using, for example, chemoselective reactions which may target specific side chains of amino acids, may be used to change the charge, polarity, hydrophobicity and potential of hydrogen bonds inside the receptor binding domains on AAV capsids.

In some embodiments of the invention, said pharmaceutical composition of the vector based on recombinant non-enveloped virus according to the present invention is intended for parenteral administration.

In some embodiments of the invention, said pharmaceutical composition of the vector based on recombinant non-enveloped virus according to the present invention is intended for intraocular administration, including intravitreal, subretinal or suprachoroidal administration, as well as for intramuscular, intravenous or subcutaneous administration.

In some embodiments of the invention, said pharmaceutical composition of the vector based on recombinant non-enveloped virus according to the present invention may be administered intravenously as an infusion.

The pharmaceutical composition of the vector based on recombinant non-enveloped virus according to the present invention may be used following dilution. To this end, the required volume of the composition is transferred from a vial to an infusion container comprising a sterile 0.9% sodium chloride solution, a sterile 5% dextrose solution, or other infusion solutions. The resulting solution is stirred by gently turning the infusion container over.

In some embodiments of the invention, the subject of treatment, or patient, is a mammal, preferably a human subject. Said subject may be either male or female, of any age.

The pharmaceutical compositions according to the present invention may be stored in any suitable container. For example, a glass or plastic container, vial, ampoule, syringe, cartridge, or bottle of the desired volume. The containers may be provided with additional means for administration, for example, droppers, auto-injectors.

The pharmaceutical composition according to the invention may be manufactured, packaged, or sold in the form of a single unit dose or a plurality of single unit doses in the form of a ready formulation. The term “single unit dose” as used herein refers to discrete quantity of a pharmaceutical composition containing a predetermined quantity of an active ingredient. The quantity of an active ingredient typically equals the dose of the active ingredient to be administered in a subject, or a convenient portion of such dose, for example, half or a third of such dose.

The pharmaceutical compositions may be administered as a single therapeutic agent or in combination with additional therapeutic agents as needed. Thus, in one embodiment, the present methods for treatment and/or prophylaxis are used in combination with administration of a therapeutically effective amount of another active agent. The other active agent may be administered before, during or following the administration of the pharmaceutical compositions according to the present invention. The other active agent may be administered as part of the present composition or, alternatively, as a separate formulation.

### EXAMPLES

The following examples are provided for better understanding of the invention. These examples are for purposes of illustration only and are not to be construed as limiting the scope of the invention in any manner.

Although the foregoing invention has been described in detail by way of illustration and example for purposes of clarity of understanding, it will be apparent to those of ordinary skill in the art in light of the teachings of this invention that certain changes and modifications may be made thereto without departing from the spirit or scope of the appended embodiments.

### Methods

### 1. Determination of Concentration of Gene of Interest (GOI) in AAV Product by PCR.

Concentration of the GOI in the AAV product was determined by quantitative polymerase chain reaction using a forward, reverse primer and a probe conjugated with the FAM dye and BHQ-1 quencher. The primers are matched to a region of the gene encoding the GOI protein. In the course of sample preparation, the sample was first treated with DNAase in order to get rid of residual plasmid DNA, then with proteinase in order to destroy the capsid. A linearized plasmid (of the corresponding GOI) was used to plot a calibration line. To plot a calibration line of log concentration as a function of cycle threshold (Ct), standards with 10-fold concentration decrements were used. The concentration of viral genomes/ml (vg/ml) in the product was determined by the calibration line of log concentration as a function of Ct.

### 2. Determination of Concentration of Viral Particles by Enzyme-Linked Immunosorbent Assay.

Concentration of viral vector particles (adeno-associated virus (AAV) capsids) was measured by means of enzyme-linked immunosorbent assay using commercial kits as follows: PROGEN AAV5 titration ELISA, PROGEN AAV6 titration ELISA, and PROGEN AAV9 titration ELISA.

High binding 96-well plates are coated with anti-AAV capsid binding monoclonal mouse antibodies recognizing the conformational epitope of a particular serotype. AAV capsid proteins bind to antibodies under temperature incubation, unbound components of the sample are removed by washing the plate multiple times.

Biotinylated antibodies that bind to AAV capsid proteins under temperature incubation of the plate were used as detecting antibodies. Unbound components are removed by washing the plate multiple times.

Streptavidin-horseradish peroxidase conjugate was applied to the plate wells to detect the color. Streptavidin binds to biotin, unbound components were removed by washing the plate multiple times.

Tetramethylbenzidine acts as a chromogen. Interaction of horseradish peroxidase, hydrogen peroxide and tetramethylbenzidine chromogen develops a color reaction. ELISA reaction was stopped by adding a stop solution (1 normal H₂SO4), and the optical density of the solutions in the wells was measured at a wavelength of 450 nm. The concentration of viral vector particles in the product was determined by a calibration graph plotted using optical density data of standard solutions with known concentration of AAV particles.

### 3 Determination of Functional Activity of the Product Based on Recombinant Adeno-Associated Viral Vector Comprising the GFP Gene.

Transduction activity of the product based on the recombinant adeno-associated viral vector comprising the GFP gene was measured by GFP expression in product-induced CHO-K1 cells.

For transduction, on the day of analysis, the CHO-K1 cells were plated into 24-well plates in a growth medium with an antibiotic (MQD-medium for quantitative determination) at the rate of 1×10⁴ cells per cm² (19,000 cells per well) and incubated at a temperature of +37° C. in an atmosphere with 5% CO₂ for 4-8 h for attachment of the cells to plastic surface. The test samples in 3 doses, 3 replications for each dose, were then introduced into the wells with cells. Product dosages (MOI) for each AAV serotype were pre-selected so as to produce, following transduction, about 5, 10 and 20% of GFP-positive cells, respectively. Product volume required for transduction of a single well was calculated using the following formula:

$$V = MOI \times S \times KC ,$$

where V is the required product volume per well (ml), S is the area of a single well (cm2), MOI is the required dose of the product, multiplicity of infection (vg/cl), K is cell seeding density in the wells (cells/cm²), C is the concentration of genome-comprising capsids in the product (vg/ml).

The plates with transduced cells were incubated in a CO₂ incubator (5% CO 2, +37° C.) for 2-3 days. Following the incubation period, culture fluid was collected from the wells, the cells were washed with Hanks solution, and the cells were removed from the plastic using TrypLE solution (at 150 μl/well). Following detachment of the cells from the plastic, Hanks solution (1:1 with TrypLE) was introduced into the wells, the cells were collected into 1.5 ml microtubes and centrifuged for 5 min at 300 g. The cell precipitate was resuspended in 150 μl of Hanks solution. 50 ml of a working dye solution for determination of viable cells in Hanks solution was added to each sample, except for 1 isotype control well, the cells were incubated for 20 minutes in the dark at room temperature. The stained cells were centrifuged for 5 minutes at 300 g, the supernatant was carefully selected and 100 μl of BD Cytofix fixing buffer was added to each sample. The cells were resuspended, and the entire volume of the resulting cell suspension was transferred into a 96-well V-bottom plate for flow cytometer, the plate with cells was incubated for 20 minutes in the dark at room temperature. Following the incubation period, the plate was centrifuged for 5 minutes at 1200 rpm, a fixation buffer was collected, and the cells were washed with Stain Buffer (PBS with 0.1% Sodium azide and 0.5% BSA) at 100 μl/well. The plate was centrifuged again and the buffer was collected, following which Stain Buffer was introduced at 150 μl/well and the cells were carefully resuspended.

The cells were analyzed on a flow cytometer. Cell viability and GFP expression were evaluated. The value of FA (functional activity) or TU (transduction unit)/ml was calculated for each product dose according to the following formula:

$$T U = P \times N V \times D ,$$

where P is the number of GFP-positive viable cells as a percentage, N is the number of initially plated cells (N=S×K), V is the introduced volume of the product per well (ml), D is the product predilution factor.

The mean TU value for 3 sample doses was considered the final result.

### 4. Determination of Concentration of Viral Particles by Size-Exclusion High-Performance Liquid Chromatography.

The concentration of viral particles was determined by way of measuring the peak areas of test items and standards on the Agilent high-performance liquid chromatograph with a UV detector. Detector wavelength: 260 and 280 nm.
Column: TSK-gel G3000SWXL 7.8×300 mm, 5 μm, 300 Å.
Phase A: 0.1M Na2SO4 in 0.1M phosphate buffer, pH=6.7.
Injection volume: 30 μl.
Flow rate: 1 ml/min.
Column temperature: 35° C.

Detector wavelength: 260 nm (4 nm), reference wavelength (360 nm, bw 100 nm); 280 nm (4 nm), reference wavelength (360 nm, bw 100 nm).
Elution mode: isocratic.
Chromatography time: 15 min.

A peak was identified on the resulting chromatograms at a wavelength of 280 and 260 nm and the area thereof was determined. Based on the resulting peak data on chromatograms of calibration solutions, a linear regression equation was created for each of the wavelengths. Using the linear regression equation at a wavelength of 280 nm, the concentration of viral particles was calculated.

Size-exclusion high-performance liquid chromatography was used as an orthogonal method for determining the concentration of viral particles.

### 5. Preparation of Pharmaceutical Compositions.

Samples with the target content of viral genomes were prepared in Stirred Cell (Millipore) concentration cells under pressure. To this end, the initial solution formulation containing viral particles was placed in a diafiltration container, then at least 10-fold volume of an aqueous solution with the target formulation including buffering, osmotic agents and, if necessary, additional water-soluble stabilizers was introduced into the cell. Following diafiltration process, the solution was concentrated to an optical density exceeding the target density, unloaded from the instrument, the concentration of viral genomes was determined by PCR. A corresponding excipient solution was then added to the sample to produce a solution with target level of viral genomes.

Also, samples of solutions containing viral particles were prepared in Pellicon (Millipore) cassettes in the tangential flow mode. To this end, the initial solution formulation was placed in a diafiltration tank, then at least 10-fold volume of the solution with the target formulation comprising buffering, osmotic agents, and, if necessary, additional water-soluble stabilizers was supplied to the system. The concentrate of osmotic agents and water-soluble stabilizers may be added following diafiltration. Following the diafiltration process, the solution was concentrated to an optical density exceeding the target density, unloaded from the system and the exact values of the optical density and concentration of viral genomes were determined. A corresponding excipient solution was then added to the sample to produce a solution with target level of viral genomes.

Prior to aseptic filling into the final container (for example, a glass/plastic vessel, vial or syringe), the solution was filtered under aseptic conditions using a 0.22 μm membrane.

### 6. Stability Study.

The test samples were divided into several aliquots and placed into distinct sterile glass vials: 1 vial for each control point was placed in a thermostat, incubated at 25° C. for 4 weeks and at (5±3° C.) for 6 months, with periodic collection at control points according to the schedule. While being collected at control points and following storage, the vials were removed from a thermostat or refrigerator and transferred for analysis.

### 7 Thermal Stress.

Test samples were divided into three aliquots and placed into glass vials: one vial for each formulation was stored in a refrigerator at (5±3° C.), the remaining vials were placed in a thermostat and incubated at a given temperature for a given period of time. When being collected at control points or following heating, the vials were removed from the thermostat, kept at room temperature for about 15 minutes, and transferred for analysis.

### 8. Shaking.

Test samples were divided into 2 aliquots and placed into glass vials: 1 vial per formulation was stored in a refrigerator at (5±3° C.), the remaining vials were placed into a thermal shaker and shaken at a speed of 800 rpm at (5±3° C.) for a given period of time. While being collected at control points or following stress, the vials were removed from the thermal shaker and transferred for analysis.

### 9 Freeze-Thaw.

Test samples were divided into two aliquots and added to glass vials: the vials were placed in a freezer and stored at minus 20° C. or below. Most gene therapy products are stored at a temperature of not more than minus 70° C.; the temperature of not more than minus 20° C. has been chosen as the “worst case” because in such method of storage, the sample is stored at a temperature above the glass transition temperature, thus subjecting the sample to additional stress similar to that occurring under freezing. Following complete freezing of the sample (after 8-16 hours), the vials were removed from the freezer, kept at room temperature until the contents were completely thawed and, if necessary, the freeze-thaw cycle was repeated. The samples were transferred for analysis following the a given number of freeze-thaw cycles.

### 10. Isothermal Study by Dynamic Light Scattering.

Colloidal stability of test samples was analyzed using the DynaPro Plate Reader II instrument. 35 μl of each test sample was added to the wells of a 384-well black polymer plate with an optically clear bottom, thereafter the plate was sealed with a heat-resistant self-adhesive film. The sealed plate was centrifuged for 3 minutes at 3000 rpm and a temperature of not less than 20° C. The measurement was carried out at a given temperature for a given time.

### Measurement Settings:

Scattered light intensity at θ (angle of scattered light measurement)=158°.
Number of measurements per replicate—3.
Time per measurement—5 s.

The time trend was analyzed using the Dynamics software (Wyatt, USA).

### 11. Determination of Aggregation Temperature.

Aggregation temperature was determined using DynaPro Plate Reader II. 35 μl of each test sample was added to the wells of a 384-well black polymer plate with an optically clear bottom, thereafter the plate was sealed with a heat-resistant self-adhesive film. The sealed plate was centrifuged for 3 minutes at 3000 rpm and a temperature of not less than 20° C.

### Measurement Settings:

Initial measurement temperature—25° C.
Scattered light intensity at θ=158°.
Number of measurements per replicate—3.
Time per measurement—5 s.
Heating rate—0.15° C./min.
Final temperature—80° C.

The temperature trend and aggregation point were determined using the Dynamics software (Wyatt, USA).

### 12. Determination of Melting Point of Capsid Proteins.

The melting point of the capsid proteins of recombinant adeno-associated viruses was determined by differential scanning fluorimetry. 5 μl of diluted SyproOrange fluorescent stain (Thermo Fisher Scientific, USA) specifically binding to hydrophobic sites of protein molecule was added to 45 μl of the test sample, until 1000-fold final dilution of the stain. The resulting mixture was transferred to a 96-well PCR plate, sealed with a self-adhesive heat-resistant film, centrifuged for 3 minutes at 3000 rpm and placed in an amplifier with a fluorimetric detector. Heating was carried out from 25 to 85° C., the temperature increment was 0.15° C., fluorescence during heating was detected in accordance with the manufacturer's instructions. CFX Manager (Bio-Rad, USA) software was used to process the results.

### 13. Determination of DNA Extraction Temperature.

Temperature of DNA extraction from capsids of vectors based on recombinant adeno-associated viruses was determined by differential scanning fluorimetry. 5 μl of diluted SybrGOLD fluorescent stain (Thermo Fisher Scientific, USA) specifically binding to nucleic acids was added to 45 μl of the test sample, until 2000-fold final dilution of the stain. The resulting mixture was transferred to a 96-well PCR plate, sealed with a self-adhesive heat-resistant film, centrifuged for 3 minutes at 3000 rpm and placed in an amplifier with a fluorimetric detector. Heating was carried out from 25 to 85° C., the temperature increment was 0.15° C., fluorescence during heating was detected in accordance with the manufacturer's instructions. CFX Manager (Bio-Rad, USA) software was used to process the results.

### 14. Determination of Samples Homogeneity by Dynamic Light Scattering.

Samples homogeneity was determined by dynamic light scattering using DynaPro Plate Reader II. 35 μl of each test sample was added to the wells of a 384-well black polymer plate with an optically clear bottom, thereafter the plate was sealed with a heat-resistant self-adhesive film. The sealed plate was centrifuged for 3 minutes at 3000 rpm and a temperature of not less than 20° C.

### Measurement Settings:

Measurement temperature: 25° C.
Keeping for 1 minute at the temperature before starting the measurement.
Scattered light intensity at θ=158°.
Number of measurements per replicate—10.
Time per measurement—5 s.

Samples size and polydispersity were determined using the Dynamics software (Wyatt, USA).

### Example 1. Selection of Excipient Formulation

The study aims at assessing the possibility of storing the recombinant adeno-associated virus vector products in liquid form at temperature conditions of (5±3)° C., as well as at selecting an optimal excipient formulation to further study the stability of products with various serotypes.

The excipients solution must provide for stability of the viral vector under expected storage conditions.

The following excipients were subjected to screening: tris(hydroxymethyl)aminomethane (buffering agent, maintenance of required pH level), sodium chloride (stabilizer, osmolyte), magnesium chloride hexahydrate (stabilizer), trehalose dihydrate (stabilizer, osmolyte, cryoprotectant), poloxamer p188 (solubilizer), L-histidine (buffering agent, maintenance of required PH level, stabilizer), L-arginine (buffering agent, maintenance of required PH level, stabilizer), L-proline (stabilizer).

Excipient formulations for test pharmaceutical compositions are shown in Table 1.

TABLE 1
Test pharmaceutical compositions

| No. | Excipient formulation | | | pH |
|---|---|---|---|---|
| 1 | L-histidine | 2.79 | mg/ml | 8.2 |
| | L-arginine | 0.349 | mg/ml | |
| | Sodium chloride | 4.4 | mg/ml | |
| | Magnesium chloride hexahydrate | 0.203 | mg/ml | |
| | Trehalose dihydrate | 50 | mg/ml | |
| | Poloxamer 188 | 0.05 | mg/ml | |
| 2 | L-histidine | 2.79 | mg/ml | 8.2 |
| | L-arginine | 0.349 | mg/ml | |
| | Sodium chloride | 11.7 | mg/ml | |
| | Magnesium chloride hexahydrate | 0.203 | mg/ml | |
| | Poloxamer 188 | 0.05 | mg/ml | |
| 3 | L-histidine | 2.79 | mg/ml | 8.2 |
| | L-arginine | 0.349 | mg/ml | |
| | L-proline | 5 | mg/ml | |
| | Sodium chloride | 4.4 | mg/ml | |
| | Magnesium chloride hexahydrate | 0.203 | mg/ml | |
| | Trehalose dihydrate | 50 | mg/ml | |
| | Poloxamer 188 | 0.05 | mg/ml | |
| 4 | Tris(hydroxymethyl)aminomethane | 2.42 | mg/ml | 8.0 |
| | Sodium chloride | 4.4 | mg/ml | |
| | Magnesium chloride hexahydrate | 0.203 | mg/ml | |
| | Trehalose dihydrate | 50 | mg/ml | |
| | Poloxamer 188 | 0.05 | mg/ml | |
| | Hydrochloric acid | to pH 8.0 | | |
| 5 | Tris(hydroxymethyl)aminomethane | 2.42 | mg/ml | 8.0 |
| | Sodium chloride | 11.7 | mg/ml | |
| | Magnesium Chloride hexahydrate | 0.203 | mg/ml | |
| | Poloxamer 188 | 0.05 | mg/ml | |
| | Hydrochloric acid | to pH 8.0 | | |
| 6 | Tris(hydroxymethyl)aminomethane | 2.42 | mg/ml | 8.0 |
| | L-proline | 5 | mg/ml | |
| | Sodium chloride | 4.4 | mg/ml | |
| | Magnesium chloride hexahydrate | 0.203 | mg/ml | |
| | Trehalose dihydrate | 50 | mg/ml | |
| | Poloxamer 188 | 0.05 | mg/ml | |

| | |
|---|---|
| Hydrochloric acid | to pH 8.0 |

The study was carried out for samples comprising about 1*10¹⁰ vg/ml of vectors based on rAAV serotype 5 bearing the GOI. Samples with various excipient formulations were prepared by diafiltration in accordance with method 5. In order to identify the effect of various excipients on the vectors stability, samples in test formulations were subjected to stresses in accordance with methods 7, 8, 9. Before and following stress, the samples were analyzed in accordance with methods 1, 2, 3, 4. The results are shown in Tables 2-5.

TABLE 2
Results of quality control of initial control formulations

| | Results | | | |
|---|---|---|---|---|
| | PCR, | ELISA, | Activity, | SE HPLC, |
| Formulation | vg/ml | particles/ml | FA/ml | vg/ml |
| Formulation 1 | 4.3 × 10¹⁰ | 2.2 × 10¹⁰ | 1.9 × 10⁵ | 3.2 × 10¹⁰ |
| Formulation 2 | 5.2 × 10¹⁰ | 2.1 × 10¹⁰ | 1.6 × 10⁵ | 4.8 × 10¹⁰ |
| Formulation 3 | 5.5 × 10¹⁰ | 2.3 × 10¹⁰ | 1.7 × 10⁵ | 3.3 × 10¹⁰ |
| Formulation 4 | 2.5 × 10¹⁰ | 1.3 × 10¹⁰ | 1.1 × 10⁵ | 1.9 × 10¹⁰ |
| Formulation 5 | 2.5 × 10¹⁰ | 1.2 × 10¹⁰ | 1.0 × 10⁵ | 2.0 × 10¹⁰ |
| Formulation 6 | 2.4 × 10¹⁰ | 1.2 × 10¹⁰ | 1.1 × 10⁵ | 1.9 × 10¹⁰ |

TABLE 3
Results of quality control of formulations following shaking

| | Results | | |
|---|---|---|---|
| Formulation | PCR, vg/ml | ELISA, particles/ml | SE HPLC, vg/ml |
| Formulation 1 | 3.9 × 10¹⁰ | 1.6 × 10¹⁰ | 3.6 × 10¹⁰ |
| Formulation 2 | 3.8 × 10¹⁰ | 1.7 × 10¹⁰ | 3.6 × 10¹⁰ |
| Formulation 3 | 3.6 × 10¹⁰ | 1.3 × 10¹⁰ | 3.5 × 10¹⁰ |
| Formulation 4 | 1.1 × 10¹⁰ | 1.0 × 10¹⁰ | 1.9 × 10¹⁰ |
| Formulation 5 | 1.2 × 10¹⁰ | 7.4 × 10⁹ | 1.9 × 10¹⁰ |
| Formulation 6 | 1.0 × 10⁷ | 6.8 × 10⁹ | 1.9 × 10¹⁰ |

TABLE 4
Results of quality control of formulations
following thermal stress (50° C.)

| | Results | | | |
|---|---|---|---|---|
| | 48 h | | 72 h | |
| | PCR, | ELISA, | PCR, | ELISA, |
| Formulation | vg/ml | particles/ml | vg/ml | particles/ml |
| Formulation 1 | 5.7 × 10⁹ | 2.6 × 10⁹ | 4.8 × 10⁹ | 1.3 × 10⁹ |
| Formulation 2 | 6.2 × 10⁹ | 2.7 × 10⁹ | 5.1 × 10⁹ | 1.9 × 10⁹ |
| Formulation 3 | 4.3 × 10⁹ | 2.3 × 10⁹ | 3.6 × 10⁹ | 1.5 × 10⁹ |
| Formulation 4 | 2.3 × 10⁸ | below the | 8.3 × 10⁷ | below LOQ |
| | | limit of | | |
| | | quantification | | |
| | | (LOQ) | | |
| Formulation 5 | 2.8 × 10⁸ | below LOQ | 6.9 × 10⁷ | below LOQ |
| Formulation 6 | 2.2 × 10⁸ | below LOQ | 1.5 × 10⁸ | below LOQ |

TABLE 5
Results of quality control of formulations following freeze-thaw

| | Results | | | | | | | |
|---|---|---|---|---|---|---|---|---|
| | 1 cycle | | | 2 cycles | | | 3 cycles | |
| | | ELISA, | SE | | ELISA, | SE | | ELISA, |
| Formu- | PCR, | particles/ | HPLC, | PCR, | particles/ | HPLC, | PCR, | particles/ |
| lation | vg/ml | ml | vg/ml | vg/ml | ml | vg/ml | vg/ml | ml |
| Formu- | 4.3 × 10¹⁰ | 1.3 × 10¹⁰ | 3.5 × 10¹⁰ | 3.2 × 10¹⁰ | 1.2 × 10¹⁰ | 3.5 × 10¹⁰ | 2.9 × 10¹⁰ | 1.8 × 10¹⁰ |
| lation 1 | | | | | | | | |
| Formu- | 3.5 × 10¹⁰ | 1.7 × 10¹⁰ | 3.7 × 10¹⁰ | 3.9 × 10¹⁰ | 1.3 × 10¹⁰ | 3.6 × 10¹⁰ | 3.3 × 10¹⁰ | 2.0 × 10¹⁰ |
| lation 2 | | | | | | | | |
| Formu- | 3.1 × 10¹⁰ | 1.2 × 10¹⁰ | 3.3 × 10¹⁰ | 4.1 × 10¹⁰ | 1.3 × 10¹⁰ | 3.5 × 10¹⁰ | 2.8 × 10¹⁰ | 1.8 × 10¹⁰ |
| lation 3 | | | | | | | | |
| Formu- | 9.4 × 10⁹ | 5.8 × 10⁹ | 2.2 × 10¹⁰ | 9.4 × 10⁹ | 7.0 × 10⁹ | 2.1 × 10¹⁰ | 1.3 × 10¹⁰ | 1.1 × 10¹⁰ |
| lation 4 | | | | | | | | |
| Formu- | 1.4 × 10¹⁰ | 6.9 × 10⁹ | 2.2 × 10¹⁰ | 1.1 × 10¹⁰ | 9.6 × 10⁹ | 1.7 × 10¹⁰ | 1.6 × 10¹⁰ | 1.1 × 10¹⁰ |
| lation 5 | | | | | | | | |
| Formu- | 1.1 × 10¹⁰ | 6.5 × 10⁹ | 1.7 × 10¹⁰ | 1.2 × 10¹⁰ | 9.2 × 10⁹ | 1.9 × 10¹⁰ | 3.4 × 10¹⁰ | 1.1 × 10¹⁰ |
| lation 6 | | | | | | | | |

The stress panel surprisingly revealed a positive effect of the use of histidine-arginine buffer solution on AAV stability. The formulation including histidine-arginine buffer solution was surprisingly found to exhibit high thermal stability. The pharmaceutical compositions based on the given buffer system showed the smallest decrease in the concentration of vector particles as analyzed by ELISA and PCR methods following 50° C. thermal stress.

Also, formulation 2 shown in Table 1 was subjected to accelerated stability studies in accordance with method 6. Samples at control points were analyzed in accordance with methods 1, 2, 3. The results of the analysis are shown in Tables 6-8.

TABLE 8
Results of quality control of formulation 2 in terms of activity
following storage at (5 ± 3) ° C.

| | Results of control of activity | | | |
|---|---|---|---|---|
| | following storage at (5 ± 3) ° C. | | | |
| | 1 month | 2 months | 3 months | 6 months |
| | Activity, | Activity, | Activity, | Activity, |
| Formulation | FA/ml | FA/ml | FA/ml | FA/ml |
| Formulation 2 | 1.7 × 10⁵ | 1.6 × 10⁵ | 2.2 × 10⁵ | 1.9 × 10⁵ |

In accordance with the resulting data of stability for 6 months at (5±3° C.) and for 1 month at (25±2° C.), formulation 2 showed good stability throughout the storage period.

### Example 2. Study of Pharmaceutical Compositions for Vectors Based on Recombinant Adeno-Associated Virus Serotype 9

The study used 5 pharmaceutical compositions shown in Table 9 and comprising the vector based on recombinant adeno-associated virus serotype 9 consisting mainly of empty capsids, at two concentrations as follows: 1*10¹² and 1*10¹³ particles/ml. Test samples were produced by diafiltration in accordance with method 5.

TABLE 9
Test pharmaceutical compositions

| Designation | Formulation | | | pH |
|---|---|---|---|---|
| HisArg | L-histidine | 2.79 | mg/ml | 8.2 |
| AAV9 | L-arginine | 0.348 | mg/ml | |
| | Sodium chloride | 11.7 | mg/ml | |
| | Magnesium chloride | 0.203 | mg/ml | |
| | Poloxamer 188 | 0.1 | mg/ml | |
| His AAV9 | L-histidine | 1.552 | mg/ml | 7.5 |
| | L-glycine | 3.754 | mg/ml | |
| | Sodium chloride | 5.844 | mg/ml | |
| | Trehalose dihydrate | 50 | mg/ml | |
| | Poloxamer 80 | 0.05 | mg/ml | |
| Tris AAV9 | Tris(hydroxymethyl)aminomethane | 4.846 | mg/ml | 8.0 |
| | Sodium chloride | 23.376 | mg/ml | |
| | Magnesium chloride | 0.407 | mg/ml | |
| | Hydrochloric acid | to pH 8.0 | | |
| | Poloxamer 188 | 0.05 | mg/ml | |
| PBS AAV9 | Sodium dihydrophosphate | 0.708 | mg/ml | 7.4 |
| | monohydrate | | | |
| | Sodium hydrophosphate | 1.305 | mg/ml | |
| | heptahydrate | | | |
| | Sodium chloride | 8.006 | mg/ml | |
| | Potassium chloride | 0.201 | mg/ml | |
| | Polysorbate 80 | 0.05 | mg/ml | |
| PhosCitr | Sodium hydrophosphate | 2.839 | mg/ml | 7.5 |
| AAV9 | Sodium chloride | 5.844 | mg/ml | |
| | Trehalose dihydrate | 50.0 | mg/ml | |
| | L-glycine | 3.754 | mg/ml | |
| | Polysorbate 80 | 0.025 | mg/ml | |

| | |
|---|---|
| Citric acid | to pH 7.5 |

Study of colloidal stability at increased temperature.

In order to measure the colloidal stability, we determined aggregation temperature of vectors based on recombinant adeno-associated viral particles in 5 formulations in accordance with method 11. The results are shown in Table 10.

TABLE 10
Aggregation temperature

| Pharmaceutical composition | Aggregation temperature, ° C. |
|---|---|
| HisArg AAV9 | 76.0 |
| His AAV9 | 52.2 |
| Tris AAV9 | 74.7 |
| PBS AAV9 | 75.9 |
| PhosCitr AAV9 | 61.0 |

Also, we studied thermal stability by thermal stress in accordance with method 7. Before and following subjecting to a temperature of 65° C. for 96 hours, samples homogeneity was analyzed by dynamic light scattering in accordance with method 14. The results are shown in Table 11.

TABLE 11
Results of colloidal stability study

| | | | | Thermal stress | |
|---|---|---|---|---|---|
| | | Initial control | | 65° C., 96 hours | |
| Pharmaceutical | Concentration, | | Mean | | Mean |
| composition | particles/ml | Polydispersity, % | radius, nm | Polydispersity, % | radius, nm |
| HisArg AAV9 | 10¹³ | 13.3 | 18.6 | Polydisperse sample | 32.7 |
| His AAV9 | | 24.2 | 22 | Polydisperse sample | 80.8 |
| Tris AAV9 | | 14.4 | 19 | Polydisperse sample | 72.6 |
| PBS AAV9 | | 18.5 | 19.1 | Polydisperse sample | 54.5 |
| PhosCitr AAV9 | | 21.1 | 21.5 | Polydisperse sample | 57.5 |
| HisArg AAV9 | 10¹² | 11.75 | 18.2 | Polydisperse sample | 18.2 |
| His AAV9 | | 24.2 | 20.8 | Polydisperse sample | 63.7 |
| Tris AAV9 | | 19.5 | 19.9 | Polydisperse sample | 18.8 |
| PBS AAV9 | | 15.8 | 18.6 | Polydisperse sample | 39.6 |
| PhosCitr AAV9 | | 23.15 | 20.6 | Polydisperse sample | 30 |

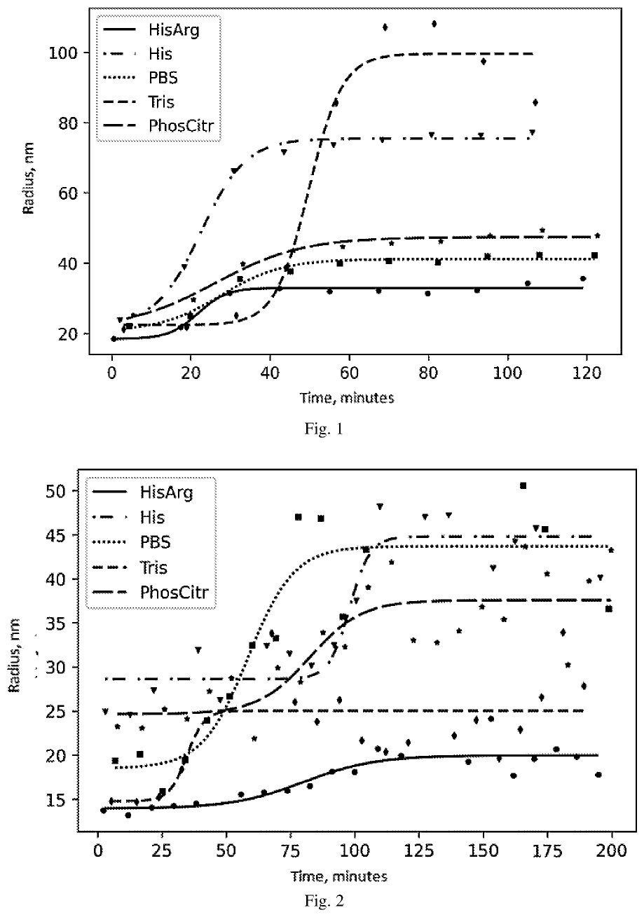

In addition, samples were controlled while subjecting to increased temperature of 65° C. for 120 minutes by dynamic light scattering in accordance with method 10. The data is shown in FIG. 1.

The pharmaceutical composition HisArg AAV9 demonstrated the best properties in terms of colloidal stability at increased temperature. The given pharmaceutical composition demonstrated the highest aggregation temperature, 76.0° C., the smallest mean radius of particles while being subjected to increased temperature and following the thermal stress as well.

Study of colloidal stability under multiple freeze-thaw cycles.

The study of the effect of multiple freeze-thaw cycles on colloidal stability of the vectors based on recombinant adeno-associated viral particles was carried out in accordance with method 9, at two concentrations of test samples. Before and following stress, samples homogeneity was analyzed by dynamic light scattering in accordance with method 14. The results are shown in Table 12.

TABLE 12
Results of colloidal stability study under multiple freeze-thaw cycles

| | | Initial control | | 5 freeze-thaw cycles | |
|---|---|---|---|---|---|
| Pharmaceutical | Concentration, | | Mean | | Mean |
| composition | particles/ml | Polydispersity, % | radius, nm | Polydispersity, % | radius, nm |
| HisArg AAV9 | 10¹³ | 13.3 | 18.6 | 11.8 | 18.3 |
| His AAV9 | | 24.2 | 22 | 25.5 | 22.3 |
| Tris AAV9 | | 14.4 | 19 | 14 | 18.8 |
| PBS AAV9 | | 18.5 | 19.1 | 18.4 | 19.5 |
| PhosCitr AAV9 | | 21.1 | 21.5 | 21.8 | 22 |
| HisArg AAV9 | 10¹² | 11.8 | 18.2 | 16 | 17.9 |
| His AAV9 | | 24.2 | 20.8 | 23.2 | 21.1 |
| Tris AAV9 | | 19.5 | 19.9 | 12.2 | 18.4 |
| PBS AAV9 | | 15.8 | 18.6 | 18.1 | 18.5 |
| PhosCitr AAV9 | | 23.15 | 20.6 | 21.2 | 20.2 |

The pharmaceutical composition HisArg AAV9 has a minimum radius and polydispersity level, indicating a reduced aggregation level under freezing as compared to other solutions.

### Example 3. Study of Pharmaceutical Compositions for Vectors Based on Recombinant Adeno-Associated Virus Serotype 6

The study used 5 pharmaceutical compositions comprising material of the vector based on rAAV serotype 6 bearing the GOI. The test pharmaceutical compositions are listed in Table 13. Test samples were produced by diafiltration in accordance with method 5.

TABLE 13
Test pharmaceutical compositions

| Designation | Formulation | | | pH |
|---|---|---|---|---|
| HisArg | L-histidine | 2.79 | mg/ml | 8.2 |
| AAV6 | L-arginine | 0.348 | mg/ml | |
| | Sodium chloride | 11.7 | mg/ml | |
| | Magnesium chloride | 0.203 | mg/ml | |
| | Poloxamer 188 | 0.1 | mg/ml | |
| His AAV6 | L-histidine | 1.552 | mg/ml | 7.5 |
| | L-glycine | 3.754 | mg/ml | |
| | Sodium chloride | 5.844 | mg/ml | |
| | Trehalose dihydrate | 50 | mg/ml | |
| | Poloxamer 80 | 0.05 | mg/ml | |
| Tris AAV6 | Tris(hydroxymethyl)aminomethane | 4.846 | mg/ml | 8.0 |
| | Sodium chloride | 23.376 | mg/ml | |
| | Magnesium chloride | 0.407 | mg/ml | |
| | Hydrochloric acid | to pH 8.0 | | |
| | Poloxamer 188 | 0.05 | mg/ml | |
| PBS AAV6 | Sodium dihydrophosphate | 0.708 | mg/ml | 7.4 |
| | monohydrate | | | |
| | Sodium hydrophosphate | 1.305 | mg/ml | |
| | heptahydrate | | | |
| | Sodium chloride | 8.006 | mg/ml | |
| | Potassium chloride | 0.201 | mg/ml | |
| | Polysorbate 80 | 0.05 | mg/ml | |
| PhosCitr | Sodium hydrophosphate | 2.839 | mg/ml | 7.5 |
| AAV6 | Sodium chloride | 5.844 | mg/ml | |
| | Trehalose dihydrate | 50.0 | mg/ml | |
| | L-glycine | 3.754 | mg/ml | |
| | Polysorbate 80 | 0.025 | mg/ml | |

| | |
|---|---|
| Citric acid | to pH 7.5 |

Study of colloidal stability at increased temperature.

In order to measure the colloidal stability, we determined aggregation temperature of vectors based on recombinant adeno-associated viral particles in 5 formulations in accordance with method 11. We used pharmaceutical compositions with viral vectors at a concentration of 1*10¹² vg/ml. The results are shown in Table 14. The results were analyzed using the Microsoft Excel heat-mapping tool.

TABLE 14
Aggregation temperature

| Pharmaceutical composition | Aggregation temperature, ° C. |
|---|---|
| HisArg AAV6 | 72.9 |
| His AAV6 | 72.3 |
| Tris AAV6 | 72.6 |
| PBS AAV6 | 66.2 |
| PhosCitr AAV6 | 40.8 |

The pharmaceutical composition HisArg AAV6 demonstrated the best colloidal stability properties at increased temperature. The given pharmaceutical composition demonstrated the highest aggregation temperature, 72.9° C.

Study of conformational and chemical stability of pharmaceutical composition HisArg AAV6.

The conformational and chemical stability studies were carried out using recombinant adeno-associated virus serotype 6 vector material at a concentration of 1*10¹² vg/ml by way of determining the denaturation temperature of capsid proteins in accordance with method 12 and the temperature of DNA extraction from capsids in accordance with method 13. The results are shown in Table 17.

TABLE 17
Results of conformational and chemical stability studies

| Pharmaceutical | DNA extraction | Capsid protein denaturation |
|---|---|---|
| composition | temperature, ° C. | temperature, ° C. |
| HisArg AAV6 | 53.1 | 76.5 |

The pharmaceutical composition HisArg AAV6 demonstrated high conformational and chemical stability, with high DNA extraction temperature and high capsid protein denaturation temperature.

### Example 4. Study of Pharmaceutical Compositions for Vectors Based on Recombinant Adeno-Associated Virus Serotype 5

The study used 5 pharmaceutical compositions comprising material of the vector based on rAAV serotype 5 bearing the GOI. The test pharmaceutical compositions are listed in Table 18. Test samples were produced by diafiltration in accordance with method 5.

TABLE 18
Test pharmaceutical compositions

| Designation | Formulation | | | pH |
|---|---|---|---|---|
| HisArg | L-histidine | 2.79 | mg/ml | 8.2 |
| AAV5 | L-arginine | 0.348 | mg/ml | |
| | Sodium chloride | 11.7 | mg/ml | |
| | Magnesium chloride | 0.203 | mg/ml | |
| | Poloxamer 188 | 0.1 | mg/ml | |
| His AAV5 | L-histidine | 1.552 | mg/ml | 7.5 |
| | L-glycine | 3.754 | mg/ml | |
| | Sodium chloride | 5.844 | mg/ml | |
| | Trehalose dihydrate | 50 | mg/ml | |
| | Poloxamer 80 | 0.05 | mg/ml | |
| Tris AAV5 | Tris(hydroxymethyl)aminomethane | 4.846 | mg/ml | 8.0 |
| | Sodium chloride | 23.376 | mg/ml | |
| | Magnesium chloride | 0.407 | mg/ml | |
| | Hydrochloric acid | to pH 8.0 | | |
| | Poloxamer 188 | 0.05 | mg/ml | |
| PBS AAV5 | Sodium dihydrophosphate | 0.708 | mg/ml | 7.4 |
| | monohydrate | | | |
| | Sodium hydrophosphate | 1.305 | mg/ml | |
| | heptahydrate | | | |
| | Sodium chloride | 8.006 | mg/ml | |
| | Potassium chloride | 0.201 | mg/ml | |
| | Polysorbate 80 | 0.05 | mg/ml | |
| PhosCitr | Sodium hydrophosphate | 2.839 | mg/ml | 7.5 |
| AAV5 | Sodium chloride | 5.844 | mg/ml | |
| | Trehalose dihydrate | 50.0 | mg/ml | |
| | L-glycine | 3.754 | mg/ml | |
| | Polysorbate 80 | 0.025 | mg/ml | |

| | |
|---|---|
| Citric acid | to pH 7.5 |

Study of colloidal stability at increased temperature.

In order to measure the colloidal stability, we determined the aggregation temperature of recombinant adeno-associated viral particles in 5 formulations in accordance with method 11. We used pharmaceutical compositions with viral vectors concentration of 10¹² vg/ml. The results are shown in Table 19.

TABLE 19
Aggregation temperature

| Pharmaceutical composition | Aggregation temperature, ° C. |
|---|---|
| HisArg AAV5 | 59.3 |
| His AAV5 | 56.4 |
| Tris AAV5 | 55.2 |
| PBS AAV5 | 57.5 |
| PhosCitr AAV5 | 54.0 |

Also, we studied thermal stability by thermal stress in accordance with method 7. Before and following subjecting to a temperature of 40° C. for 96 hours, samples homogeneity was analyzed by dynamic light scattering in accordance with method 14. The results are shown in Table 20.

TABLE 20
Results of colloidal stability study

| | | | 40° C. thermal | |
|---|---|---|---|---|
| | Initial control | | stress, 96 hours | |
| Pharmaceutical | Polydis- | Mean | Polydis- | Mean |
| composition | persity, % | radius, nm | persity, % | radius, nm |
| HisArg AAV5 | 4.7 | 13.6 | 17.1 | 14.2 |
| His AAV5 | 19.0 | 27.7 | 45.7 | 32.0 |
| Tris AAV5 | 5.6 | 14.6 | 20.1 | 16.0 |
| PBS AAV5 | 23.9 | 16.7 | 18.6 | 16.5 |
| PhosCitr AAV5 | 19.9 | 19.9 | 25.1 | 19.7 |

In addition, samples were controlled while subjecting to increased temperature of 40° C. for 200 minutes by dynamic light scattering in accordance with method 10. The data is shown in FIG. 2.

The pharmaceutical composition HisArg AAV5 demonstrated the best colloidal stability properties at increased temperature. The given composition demonstrated the highest aggregation temperature, 59.3° C., the smallest mean radius of particles while being subjected to increased temperature and minimum mean radius and polydispersity following the thermal stress as well.

Study of colloidal stability under multiple freeze-thaw cycles.

The effect of multiple freeze-thaw cycles on the colloidal stability of recombinant adeno-associated viral particles was studied in accordance with method 9. Before and following stress, samples homogeneity was analyzed by dynamic light scattering in accordance with method 14. The results are shown in Table 21.

TABLE 21
Results of colloidal stability study
under multiple freeze-thaw cycles

| | Initial control | | 5 freeze-thaw cycles | |
|---|---|---|---|---|
| Pharmaceutical | Polydis- | Mean | Polydis- | Mean |
| composition | persity, % | radius, nm | persity, % | radius, nm |
| HisArg AAV5 | 4.7 | 13.6 | 8.3 | 13.7 |
| His AAV5 | 19.0 | 27.7 | 12.5 | 19.2 |
| Tris AAV5 | 5.6 | 14.6 | 8.3 | 14.2 |
| PBS AAV5 | 23.9 | 16.7 | 15.7 | 15.7 |
| PhosCitr AAV5 | 19.9 | 19.9 | 18.4 | 23.2 |

The pharmaceutical composition HisArg AAV5 demonstrated the minimum size and polydispersity among test samples.

Study of conformational and chemical stability.

The conformational and chemical stability studies were carried out using recombinant adeno-associated virus serotype 5 material at a concentration of 1*10¹³ vg/ml by way of determining the denaturation temperature of capsid proteins in accordance with method 12 and the temperature of DNA extraction from capsids in accordance with method 13. The results are shown in Table 22.

TABLE 22
Results of chemical stability study

| Pharmaceutical composition | DNA extraction temperature, ° C. |
|---|---|
| HisArg AAV5 | 55.5 |
| His AAV5 | 52.5 |
| Tris AAV5 | 50.7 |
| PBS AAV5 | 51.3 |
| PhosCitr AAV5 | 51.9 |

The pharmaceutical composition HisArg AAV5 demonstrated the highest DNA extraction temperature, indicating its high stabilizing properties. The pharmaceutical composition HisArg AAV5 also demonstrated a high capsid protein denaturation temperature, 87.0° C.

### Example 5. Study of Effect of Excipient Formulation on the Tendency of Vectors Based on Recombinant Adeno-Associated Viruses to Extract DNA

In order to study the effect of a pharmaceutical composition on the tendency to extract DNA from a capsid, we used two pharmaceutical compositions shown in Table 23. The given formulations demonstrated the best results in the study of recombinant adeno-associated serotype 5 particles at a concentration of 1*10¹³ vg/ml.

TABLE 23
Test excipient formulations

| Designation | Formulation | | | pH |
|---|---|---|---|---|
| HisArg | L-histidine | 2.79 | mg/ml | 8.2 |
| | L-arginine | 0.348 | mg/ml | |
| | Sodium chloride | 11.7 | mg/ml | |
| | Magnesium chloride | 0.203 | mg/ml | |
| | Poloxamer 188 | 0.1 | mg/ml | |
| His | L-histidine | 1.552 | mg/ml | 7.5 |
| | L-glycine | 3.754 | mg/ml | |
| | Sodium chloride | 5.844 | mg/ml | |
| | Trehalose dihydrate | 50 | mg/ml | |
| | Poloxamer 80 | 0.05 | mg/ml | |

The study was carried out using material of the vector based on rAAV serotypes 2, 5, 6, 8, 9 bearing the GOI, at a concentration of 1*10¹² vg/ml. Test samples were produced by diafiltration in accordance with method 5. The analysis was carried out in accordance with method 13.

TABLE 24
Temperature of DNA extraction from capsids

DNA extraction temperature

| Serotype | His | HisArg |
|---|---|---|
| AAV9 | 71.7 | 72.6 |
| AAV6 | 52.8 | 53.1 |
| AAV2 | 54.9 | 63.6 |
| AAV5 | 51.6 | 56.1 |
| AAV8 | 48.9 | 51.6 |

For all test vectors based on adeno-associated viral particles of various serotypes, the use of a pharmaceutical composition:

| | | |
|---|---|---|
| L-histidine | 2.79 | mg/ml |
| L-arginine | 0.348 | mg/ml |
| Sodium chloride | 11.7 | mg/ml |
| Magnesium chloride | 0.203 | mg/ml |
| Poloxamer 188 | 0.1 | mg/ml |

led to increased temperature of DNA extraction as compared to an alternative pharmaceutical composition based on a histidine buffer system, indicating better stability in DNA retention inside the capsid of test items in the given formulation.

The developed compositions may be applied to adeno-associated viruses and other types of non-enveloped viruses.

1. A pharmaceutical composition of a vector based on recombinant non-enveloped virus, comprising:
(i) a vector based on recombinant non-enveloped virus,
(ii) histidine,
(iii) arginine,
(iv) sodium chloride,
(v) magnesium chloride,
(vi) a surfactant, and
(vii) water for injection.

2. The pharmaceutical composition according to claim 1, wherein histidine is present at a concentration of 2.0-3.58 mg/ml.

3. The pharmaceutical composition according to claim 1, wherein histidine is present at a concentration of 2.3-3.2 mg/ml; or
histidine is present at a concentration of 2.6-3.0 mg/ml; or
histidine is present at a concentration of 2.7-2.8 mg/ml.

4. The pharmaceutical composition according to claim 1, wherein arginine is present at a concentration of 0.248-0.448 mg/ml.

5. The pharmaceutical composition according to claim 1, wherein arginine is present at a concentration of 0.28-0.416 mg/ml; or
arginine is present at a concentration of 0.3-0.396 mg/ml; or
arginine is present at a concentration of 0.340-0.350 mg/ml.

6. The pharmaceutical composition according to claim 1, wherein sodium chloride is present at a concentration of 8.0-15.0 mg/ml.

7. The pharmaceutical composition according to claim 1, wherein sodium chloride is present at a concentration of 9.5-13.5 mg/ml; or
sodium chloride is present at a concentration of 10.5-12.5 mg/ml; or
sodium chloride is present at a concentration of 11.0-12.0 mg/ml.

8. The pharmaceutical composition according to claim 1, wherein magnesium chloride is present at a concentration of 0.15-0.50 mg/ml.

9. The pharmaceutical composition according to claim 1, wherein magnesium chloride is present at a concentration of 0.16-0.35 mg/ml; or
magnesium chloride is present at a concentration of 0.18-0.25 mg/ml; or
magnesium chloride is present at a concentration of 0.200-0.210 mg/ml.

10. The pharmaceutical composition according to claim 1, wherein the surfactant is present at a concentration of 0.01-1.0 mg/ml.

11. The pharmaceutical composition according to claim 1, wherein the surfactant is present at a concentration of 0.01-0.5 mg/ml; or
the surfactant is present at a concentration of 0.01-0.2 mg/ml; or
the surfactant is present at a concentration of 0.03-0.15 mg/ml.

12. The pharmaceutical composition according to claim 1, comprising:

| (i) | a vector based on recombinant | | |
|---|---|---|---|
| | non-enveloped virus, | | |
| (ii) | histidine | 2.0-3.58 | mg/ml, |
| (iii) | arginine | 0.248-0.448 | mg/ml, |
| (iv) | sodium chloride | 8.0-15.0 | mg/ml, |
| (v) | magnesium chloride | 0.15-0.50 | mg/ml, |
| | | |
|---|---|---|
| (vi) | a surfactant | 0.01-1.0 mg/ml, and |
| (vii) | water for injection | ad 1.0 ml. |

13. The pharmaceutical composition according to claim 1, comprising:

| (i) | a vector based on recombinant | | |
|---|---|---|---|
| | non-enveloped virus, | | |
| (ii) | histidine | 2.3-3.2 | mg/ml, |
| (iii) | arginine | 0.28-0.416 | mg/ml, |
| (iv) | sodium chloride | 9.5-13.5 | mg/ml, |
| (v) | magnesium chloride | 0.16-0.35 | mg/ml, |
| | | |
|---|---|---|
| (vi) | a surfactant | 0.01-0.2 mg/ml, and |
| (vii) | water for injection | ad 1.0 ml. |

14. The pharmaceutical composition according to claim 1, comprising:

| (i) | a vector based on recombinant | | |
|---|---|---|---|
| | non-enveloped virus, | | |
| (ii) | histidine | 2.6-3.0 | mg/ml, |
| (iii) | arginine | 0.3-0.396 | mg/ml, |
| (iv) | sodium chloride | 10.5-12.5 | mg/ml, |
| (v) | magnesium chloride | 0.18-0.25 | mg/ml, |
| | | |
|---|---|---|
| (vi) | a surfactant | 0.03-0.15 mg/ml, and |
| (vii) | water for injection | ad 1.0 ml. |

15. The pharmaceutical composition according to claim 1, comprising:

| (i) | a vector based on recombinant | | |
|---|---|---|---|
| | non-enveloped virus, | | |
| (ii) | histidine | 2.7-2.8 | mg/ml, |
| (iii) | arginine | 0.340-0.350 | mg/ml, |
| (iv) | sodium chloride | 11.0-12.0 | mg/ml, |
| (v) | magnesium chloride | 0.200-0.210 | mg/ml, |
| | | |
|---|---|---|
| (vi) | a surfactant | 0.03-0.15 mg/ml, and |
| (vii) | water for injection | ad 1.0 ml. |

16. A pharmaceutical composition, wherein histidine is present at a concentration of 2.79 mg/ml.

17. The pharmaceutical composition according to claim 1, wherein arginine is present at a concentration of 0.348 mg/ml.

18. The pharmaceutical composition according to claim 1, wherein sodium chloride is present at a concentration of 11.7 mg/ml.

19. The pharmaceutical composition according to claim 1, wherein magnesium chloride is present at a concentration of 0.203 mg/ml.

20. The pharmaceutical composition according to claim 1, wherein the surfactant is present at a concentration of 0.05 mg/ml or 0.1 mg/ml.

21. The pharmaceutical composition according to claim 1, comprising:
(i) a vector based on recombinant non-enveloped virus,

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride | 0.203 | mg/ml, |
| (vi) | a surfactant | 0.05 | mg/ml, |
| | | |
|---|---|---|
| (vii) | water for injection | ad 1.0 ml; |

or
(i) a vector based on recombinant non-enveloped virus,

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride | 0.203 | mg/ml, |
| (vi) | a surfactant | 0.1 | mg/ml, |
| | | |
|---|---|---|
| (vii) | water for injection | ad 1.0 ml. |

22. The pharmaceutical composition according to claim 1, wherein magnesium chloride is magnesium chloride hexahydrate.

23. The pharmaceutical composition according to claim 1, wherein the surfactant is poloxamer 188.

24. The pharmaceutical composition according to claim 1, comprising:

| (i) | a vector based on recombinant | | |
|---|---|---|---|
| | non-enveloped virus, | | |
| (ii) | histidine | 2.0-3.58 | mg/ml, |
| (iii) | arginine | 0.248-0.448 | mg/ml, |
| (iv) | sodium chloride | 8.0-15.0 | mg/ml, |
| (v) | magnesium chloride being | 0.15-0.50 | mg/ml, |
| | magnesium chloride hexahydrate | | |
| (vi) | a surfactant being poloxamer 188 | 0.01-1.0 | mg/ml, |
| | | |
|---|---|---|
| (vii) | water for injection | ad 1.0 ml. |

25. The pharmaceutical composition according to claim 1, comprising:

| (i) | a vector based on recombinant | | |
|---|---|---|---|
| | non-enveloped virus, | | |
| (ii) | histidine | 2.3-3.2 | mg/ml, |
| (iii) | arginine | 0.28-0.416 | mg/ml, |
| (iv) | sodium chloride | 9.5-13.5 | mg/ml, |
| (v) | magnesium chloride being | 0.16-0.35 | mg/ml, |
| | magnesium chloride hexahydrate | | |
| | | |
|---|---|---|
| (vi) | a surfactant being poloxamer 188 | 0.01-0.2 mg/ml, and |
| (vii) | water for injection | ad 1.0 ml. |

26. The pharmaceutical composition according to claim 1, comprising:

| (i) | a vector based on recombinant | | |
|---|---|---|---|
| | non-enveloped virus, | | |
| (ii) | histidine | 2.6-3.0 | mg/ml, |
| (iii) | arginine | 0.3-0.396 | mg/ml, |
| (iv) | sodium chloride | 10.5-12.5 | mg/ml, |
| (v) | magnesium chloride being | 0.18-0.25 | mg/ml, |
| | magnesium chloride hexahydrate | | |
| | | |
|---|---|---|
| (vi) | a surfactant being poloxamer 188 | 0.03-0.15 mg/ml, and |
| (vii) | water for injection | ad 1.0 ml. |

27. The pharmaceutical composition according to claim 1, comprising:

| (i) | a vector based on recombinant | | |
|---|---|---|---|
| | non-enveloped virus, | | |
| (ii) | histidine | 2.7-2.8 | mg/ml, |
| (iii) | arginine | 0.340-0.350 | mg/ml, |
| (iv) | sodium chloride | 11.0-12.0 | mg/ml, |
| (v) | magnesium chloride being | 0.200-0.210 | mg/ml, |
| | magnesium chloride hexahydrate | | |
| | | |
|---|---|---|
| (vi) | a surfactant being poloxamer 188 | 0.03-0.15 mg/ml, and |
| (vii) | water for injection | ad 1.0 ml. |

28. The pharmaceutical composition according to claim 1, comprising:

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride being | 0.203 | mg/ml, |
| | magnesium chloride hexahydrate | | |
| (vi) | a surfactant being poloxamer 188 | 0.05 | mg/ml, |
| | | |
|---|---|---|
| (vii) | water for injection | ad 1.0 ml; |

or

| (i) | a vector based on recombinant | | |
|---|---|---|---|
| | non-enveloped virus, | | |
| (ii) | histidine | 2.79 | mg/ml, |
| (iii) | arginine | 0.348 | mg/ml, |
| (iv) | sodium chloride | 11.7 | mg/ml, |
| (v) | magnesium chloride being | 0.203 | mg/ml, |
| | magnesium chloride hexahydrate | | |
| (vi) | a surfactant being poloxamer 188 | 0.1 | mg/ml, |
| | | |
|---|---|---|
| (vii) | water for injection | ad 1.0 ml. |

29. The pharmaceutical composition according to claim 1, wherein histidine is L-histidine.

30. The pharmaceutical composition according to claim 1, wherein arginine is L-arginine.

31. The pharmaceutical composition according to claim 1, wherein the pharmaceutical composition has pH 7.0-9.0.

32. The pharmaceutical composition according to claim 1, wherein the pharmaceutical composition has pH 7.7-8.7; or
the pharmaceutical composition has pH 7.5-8.5.

33. The pharmaceutical composition according to claim 1, wherein the pharmaceutical composition has pH 8.2.

34. The pharmaceutical composition according to claim 1, wherein the vector based on recombinant non-enveloped virus is present at a concentration of 1.0*10⁹-1.0*10¹⁴ viral genomes/ml.

35. The pharmaceutical composition according to claim 1, wherein the vector based on recombinant non-enveloped virus is present at a concentration of (1.0±0.3)*10⁹ viral genomes/ml, (2.0±0.6)*10⁹ viral genomes/ml, (5.0±1.5)*10⁹ viral genomes/ml, (1.0±0.3)*10¹⁰ viral genomes/ml, (2.0±0.6)*10¹⁰ viral genomes/ml, (5.0±1.5)*10¹⁰ viral genomes/ml, (1.0±0.3)*10¹¹ viral genomes/ml, (2.0±0.6)*10¹¹ viral genomes/ml, (5.0±1.5)*10¹¹ viral genomes/ml, (1.0±0.3)*10¹² viral genomes/ml, (2.0±0.6)*10¹² viral genomes/ml, (5.0±1.5)*10¹² viral genomes/ml, (1.0±0.3)*10¹³ viral genomes/ml (2.0±0.6)*10¹³ viral genomes/ml, (5.0±1.5)*10¹³ viral genomes/ml, or (1.0=0.3)*10¹⁴ viral genomes/ml.

36. The pharmaceutical composition according to claim 1, wherein the recombinant non-enveloped virus vector is a vector based on rAAV.

37. The pharmaceutical composition according to claim 36, wherein the vector based on rAAV comprises a capsid of AAV5 serotype, AAV6 serotype, or AAV9 serotype.

38. The pharmaceutical composition according to claim 37, wherein the capsid of AAV5 serotype, AAV6 serotype, or AAV9 serotype may be a modified capsid.

39. The pharmaceutical composition according to any one of claims 1-38, which is suitable for lyophilization.
